(19) United States
(12) Patent Application Publication
(10) Pub. No.: US 2019/0083049 A1
(43) Pub. Date: Mar. 21, 2019

(54) Massively Multi-Frequency Ultrasound-Encoded Tomography

(71) Applicant: The Charles Stark Draper Laboratory, Inc., Cambridge, MA (US)

(72) Inventors: Steven J. Byrnes, Watertown, MA (US); Joseph Hollmann, Watertown, MA (US)

(21) Appl. No.: 16/134,017

(22) Filed: Sep. 18, 2018

Related U.S. Application Data
(60) Provisional application No. 62/653,646, filed on Apr. 6, 2018.
(60) Provisional application No. 62/621,100, filed on Jan. 24, 2018.
(60) Provisional application No. 62/582,391, filed on Nov. 7, 2017.
(60) Provisional application No. 62/559,779, filed on Sep. 18, 2017.

(51) Int. Cl. A61B 6/03; A61B 5/00; G02B 27/12; G01S 15/89; A61B 6/00; A61B 8/00

(52) U.S. Cl. CPC A61B 6/032; A61B 5/0097; A61B 8/4477; G01S 15/8952; A61B 6/5205; G02B 27/12

(57) ABSTRACT

A system and corresponding method are described for multi-frequency ultrasonically-encoded tomography of a target object. One or more probe inputs generate probe input signals to the target object. An ultrasound transducer array is placed on the outer surface of the target object and has multiple ultrasound transducers each generating a different time-dependent waveform to form a plurality of ultrasound input signals to a target probe volume within the target object. A photorefractive crystal mixes scattered light output signals from the target probe volume with an optical reference beam input to produce optical tomography output signals including ultrasound sum frequencies components. A photodetector senses the optical tomography output signals from the photorefractive crystal. A tomography analysis of the tomography output signals including the ultrasound sum frequencies components is performed to create a three-dimensional object map representing structural and/or functional characteristics of the target object.

This application claims priority from U.S. Provisional Patent Application 62/653,646, filed Apr. 6, 2018, and U.S. Provisional Patent Application 62/621,100, filed Jan. 24, 2018, and U.S. Provisional Patent Application 62/582,391, filed Nov. 7, 2017, and U.S. Provisional Patent Application 62/559,779, filed Sep. 18, 2017, all of which are incorporated herein by reference in their entireties.

### TECHNICAL FIELD

The present invention relates to multi-frequency arrangements for ultrasonically-encoded tomography.

### BACKGROUND ART

Tomography refers to the imaging of a target object by sections using of any kind of penetrating wave. One family of tomography techniques is variously called ultrasound-encoded tomography, ultrasound-modulated tomography, or various more specific terms as discussed below. Generally this involves some form of probe input signals (e.g., an electrical signal injected by an electrode, current induced by a changing current in a magnetic coil, microwave-frequency electromagnetic wave, near-infrared-frequency electromagnetic wave, etc.) at some input frequency ωᵢₙ, and a tomography output signal (either of the same or different form, i.e., voltage detected with an electrode, or current picked up by a magnetic coil, or microwave-frequency receiver, or near-infrared-frequency detector, or various other possibilities) which is detected, and simultaneously there is present a modulating ultrasound input signal of frequency ω_{ultrasound}. The tomography output signal includes an interaction component that is generated by interaction of the probe input signals with the ultrasound input signals, specifically, sideband frequencies ωₒᵤₜ=ωᵢₙ±ω_{ultrasound}, which is measured either directly or through heterodyne techniques, and this forms the basis for the tomography measurement. In some cases, the probe input signal has zero frequency (DC) or is not present at all, in which case ωₒᵤₜ=ω_{ultrasound}. The primary purpose of the modulating ultrasound input signal is to improve spatial resolution of the system, leading to resolution comparable to the ultrasound wavelength (perhaps 1 mm), which might be substantially better than the same technique's resolution without ultrasound encoding. Relatedly, the ultrasound tends to improve the noise-tolerance of the spatial reconstruction, and to require less prior knowledge or assumptions about the target object volume being measured.

One category of ultrasound-encoded tomography is called “ultrasound-encoded optical tomography” or “acousto-optic tomography”. This is a type of ultrasound-encoded tomography based on diffuse optical tomography. Its goal is to create high-resolution optical (visible or near-infrared) 3D images of tissues or other highly-scattering media, at one or more wavelengths. These techniques have potential applications in diagnosing injuries, functional brain imaging, fetus imaging, cancer screening, image-guided surgery, image-guided radiation therapy, and many other areas.

FIG. 1 illustrates the principle of conventional ultrasound-modulated optical tomography (see for example, “Photorefractive detection of tagged photons in ultrasound modulated optical tomography of thick biological tissues”, Ramaz et al., Optics Express 12, 5469 (2004), which is incorporated herein by reference in its entirety). Target tissue 102 such as brain tissue of a patient can be considered as a medium that is transparent to ultrasound, but highly scattering to light. A probe input light source 101, an ultrasound transducer phased array 103, and an optical sensor 105 are all placed on the target tissue 102 and operated by an optical tomography processor 106 that includes at least one hardware processor and which may be coupled to data storage memory (not shown) that is configured for storing optical tomography software and other system information and signals. The tomography processor 106 is configured to execute the optical tomography software including instructions to operate the ultrasound transducers in the ultrasound transducer array 103 to focus ultrasound waves (e.g. at 5 MHz) to an imaging volume 104, which is a particular small region in three-dimensional space in the target tissue 102 (which also can be thought of and referred to as a “voxel”). The tomography processor 106 also operates the light source 101 to provide one or more light input signals to the target tissue 102. The light input signals scatter randomly in all directions, tracing complicated paths through the target tissue 102. However, some small fraction of the light signals travel from the light source 101, through the imaging volume 104, and out to the optical sensor 105. This scattered light is modulated in intensity and/or phase at 5 MHz, effectively creating optical sidebands shifted by ±5 MHz from the optical frequency. The tomography processor 106 detects these sidebands through any of several methods—most simply digitizing the received intensity and calculating the component that oscillates at 5 MHz, but alternatively using more sophisticated detection methods such as discussed as in Ramaz et al. (above). The intensity and phase of the scattered light sidebands indicates the properties of that imaging volume 104, including its light intensity, acousto-optic coefficient, etc. After measuring one imaging volume 104, the tomography processor 106 can change the ultrasound phase pattern delivered by the ultrasound transducer array 103 to measure another imaging volume, and so on.

A non-invasive three-dimensional optical video of patient tissue such as the brain using multiple wavelengths could reveal useful information including real-time spectroscopic information of the target imaging volume, which can be used for highly-specific quantitative maps of many different bio-markers in parallel. This can represent information about tissue parameters such as blood oxygenation, glucose, clots, swelling, and neuron firing; see for example, “In Vivo Observations of Rapid Scattered Light Changes Associated with Neurophysiological Activity”, Rector et al. from book: In Vivo Optical Imaging of Brain Function, 2009, which is incorporated herein by reference in its entirety. This could lead to new diagnostic approaches for many medical conditions such as traumatic brain injury and tumors, and could also provide maps of brain activation patterns, with implications for psychiatric diagnostics, communication systems for paraplegics and others, control of prosthetics, and brain-machine interfaces more generally.

In certain spectral windows, particularly including red and near infrared (NIR), light from non-invasive external light sources can penetrate through the skin and skull into the target tissue (e.g., the brain) sufficiently to get meaningful data out. Unfortunately, red and NIR light undergoes multiple scattering which obfuscates the spatial structure of the target tissue, thus making it very challenging to get a high-resolution spatial map. There is currently no good solution to this problem.

### SUMMARY

Embodiments of the present invention are directed to computer-implemented systems for multi-frequency ultrasonically-encoded optical tomography of a target object such as a brain of a patient. One or more probe inputs are configured for generating optical probe input signals to the target object. An ultrasound transducer array is configured for placement on the outer surface of the target object and has multiple ultrasound transducers each generating a different time-dependent waveform to form multiple ultrasound input signals to a target probe volume within the target object. A photorefractive crystal is configured for mixing scattered light output signals from the target probe volume with an optical reference beam input to the photorefractive crystal to produce optical tomography output signals including ultrasound sum frequencies components. A photodetector are configured for sensing the optical tomography output signals from the photorefractive crystal, in the form of intensity modulation of the scattered light beam passing through the photorefractive crystal. Data storage memory is configured for storing optical tomography software, the optical tomography output signals, and other system information. A tomography processor includes at least one hardware processor coupled to the data storage memory and configured to execute the optical tomography software including instructions to perform acousto-optic tomography analysis of the optical tomography output signals including ultrasound sum frequencies components to create a three-dimensional object map representing structural and/or functional characteristics of the target object.

Specific embodiments may further include an auxiliary photodetector which is configured for sensing the light in the reference beam that passes straight through the photorefractive crystal including optical tomography output signals manifested as light intensity changes opposite in sign from the primary photodetector, and including ultrasound sum frequencies components, and the optical tomography software executed by the tomography processor includes instructions to perform acousto-optic tomography analysis of the optical tomography output signals from the primary and auxiliary photodetectors including ultrasound sum frequencies components. In addition or alternative, there may be an optical fiber arrangement configured for communicating the scattered light output signals from the target probe volume to the photorefractive crystal.

In specific embodiments, the different time-dependent waveforms may represent different ultrasound frequencies. The optical reference beam input may be from the one or more probe inputs generating the optical probe input signals. The optical tomography output signals may further include ultrasound difference frequencies components, and the optical tomography software executed by the tomography processor may further include instructions to perform acousto-optic tomography analysis of the ultrasound sum frequencies components and the ultrasound difference frequencies components of the optical tomography output signals. The photorefractive detector elements may be configured for operation at a speed at least four times greater than the greatest ultrasound frequency. The optical tomography software executed by the tomography processor may include instructions to perform acousto-optic tomography analysis using matched filters to create the three-dimensional object map. In addition or alternatively, the optical tomography software executed by the tomography processor may include instructions to perform acousto-optic tomography analysis using ultrasound waveform predictions that include a pressure-squared-versus-time profile and a displacement-squared-versus-time profile for each sampling point. The optical tomography software executed by the tomography processor may also include instructions to perform acousto-optic tomography analysis using supplemental optical tomography output signals having ultrasound components at the ultrasound frequencies of the ultrasound input signals. The photorefractive crystal may be made of gallium arsenide. And the ultrasound sum frequencies components specifically may include second-harmonic frequency components.

Embodiments of the present invention also include computer-implemented methods employing at least one hardware implemented computer processor for multi-frequency ultrasonically-encoded optical tomography of a target object having an outer surface; for example, the brain of a patient. The at least one hardware processor is operated to execute program instructions for:
generating optical probe input signals to the target object;
operating an ultrasound transducer array placed on the outer surface of the target object and having multiple ultrasound transducers each generating a different time-dependent waveform to form multiple ultrasound input signals to a target probe volume within the target object;
mixing scattered light output signals from the target probe volume with an optical reference beam in a photorefractive crystal so as to produce optical tomography output signals including ultrasound sum frequency components;
sensing the optical tomography output signals from the photorefractive crystal with a photodetectors; and
performing acousto-optic tomography analysis of the ultrasound sum frequency components of the optical tomography output signals to create a three-dimensional object map representing structural and/or functional characteristics of the target object.

Further specific embodiments, may also include sensing an auxiliary signal from the reference beam transmitted through the photorefractive crystal consisting of light intensity modulation signals opposite in sign from the primary signal, and including, wherein the acousto-optic tomography analysis includes the ultrasound sum frequencies components of the optical tomography output signals. Embodiments may also include the step of communicating the scattered light output signals from the target probe volume to the photorefractive crystal with an optical fiber or fiber bundle arrangement.

The different time-dependent waveforms may represent different ultrasound frequencies. The optical reference beam input may be generated by one or more probe inputs generating the optical probe input signals. The optical tomography output signals may further include ultrasound difference frequencies components, wherein the acousto-optic tomography analysis is of the ultrasound sum frequencies components and the ultrasound difference frequencies components of the optical tomography output signals. The photorefractive detector elements may be configured for operation at a speed at least four times greater than the greatest ultrasound frequency. The acousto-optic tomography analysis may use matched filters to create the three-dimensional object map. The acousto-optic tomography analysis may use ultrasound waveform predictions that include a pressure-squared-versus-time profile and displacement-squared-versus-time profile for each sampling point, and/or the acousto-optic tomography analysis may use supplemental optical tomography output signals having ultrasound components at the ultrasound frequencies of the ultrasound input signals. The photorefractive crystal may be made of gallium arsenide. And the ultrasound sum frequencies components specifically may include second-harmonic frequency components.

### BRIEF DESCRIPTION OF THE DRAWINGS

FIG. 1 illustrates the principle of conventional ultrasound-modulated optical tomography.

FIG. 2 illustrates the principle of a multi-frequency arrangement for ultrasound-modulated optical tomography.

FIG. 3 shows an arrangement for direct multi-frequency optical tomography according to an embodiment of the present invention.

FIG. 4 shows an example of acousto-optical interaction in two exemplary voxels according to an embodiment of the present invention.

FIG. 5 shows an arrangement for heterodyned multi-frequency optical tomography according to an embodiment of the present invention.

FIG. 6 shows an arrangement for heterodyned multi-frequency optical tomography using multiple wavelength input light.

FIG. 7 shows an arrangement for direct multi-frequency optical tomography using multiple wavelength input light.

FIG. 8 shows an example of the geometry for an input/sensing device according to an embodiment of the present invention.

FIG. 9 shows an embodiment of the present invention based on photorefractive detection.

### DETAILED DESCRIPTION

The discussion that follows is set forth in terms of examples of multi-frequency ultrasonically-encoded tomography that specifically perform ultrasonically-encoded optical tomography. But the skilled person will understand that the invention is not limited to such applications and includes other specific forms of ultrasonically-encoded tomography as explained later. In addition, the following discussion and examples are set forth in terms of red/infrared imaging of the brain. But the various discussed techniques may be useful for any medium which is highly scattering to light. Other specific applications include other tissues (e.g. breast cancer diagnostics), imaging in turbid water, generating a 3D refractive index map of water to infer its temperature profile, microwave probing of the brain and other tissues, microwave probing of pipes and other infrastructure and geological features, and so on. Also, the discussion is set forth using terms like “light” and “optical”, it will be understood to refer generically to electromagnetic radiation, which could be any specific frequency from ultraviolet to radio.

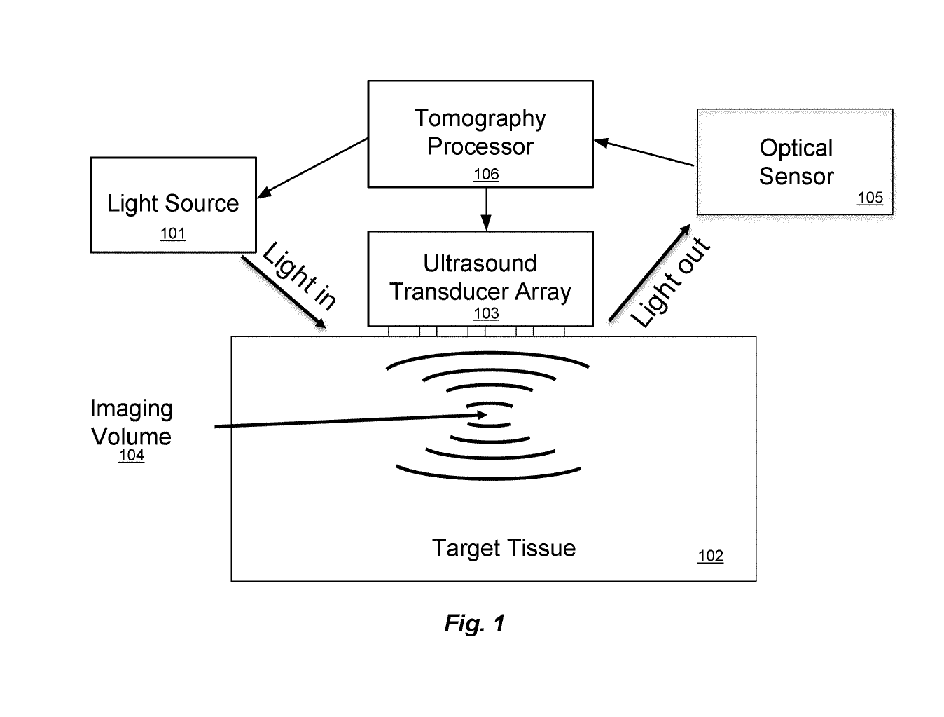

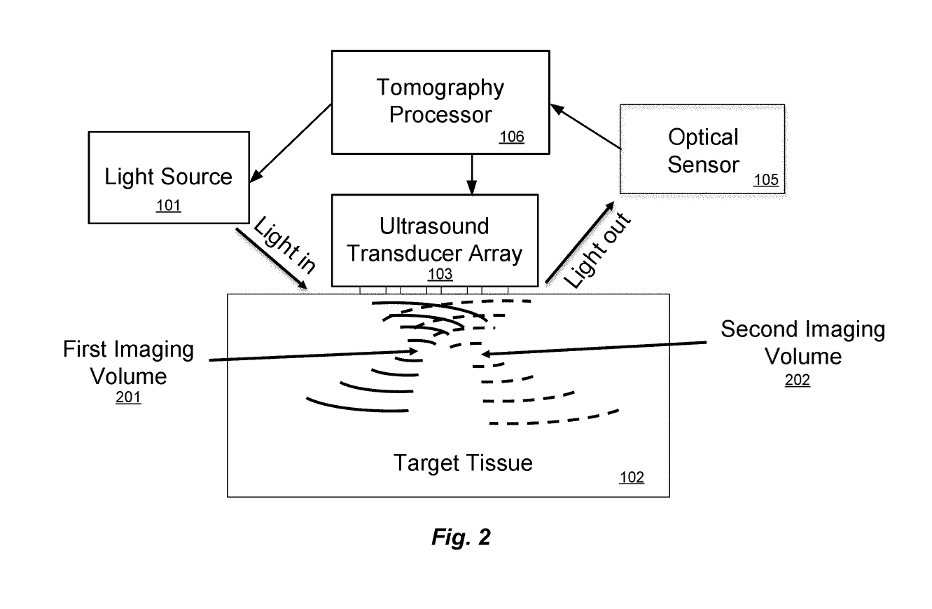

FIG. 2 illustrates the operating principle for a multi-frequency arrangement for ultra-sound modulated optical tomography, derived from the system that was discussed with respect to FIG. 1. Each transducer element of the ultrasound transducer array 106 can be considered as being attached to an arbitrary waveform generator, as an example. The tomography processor 106 can then simultaneously focus 5 MHz ultrasound into a first target imaging volume 201, and 5.1 MHz ultrasound into a different second target imaging volume 202, simply by superimposing the corresponding ultrasound waveform patterns from the ultrasound transducer array 103. The optical sensor 105 and the tomography processor 106 then can simultaneously monitor the 5 MHz and 5.1 MHz scattered light sidebands to simultaneously determine information from each of these imaging volumes. This approach can be extended into as many simultaneous imaging volumes as desired, at least up to the resolution limitations imposed by the ultrasound wavelength.

The multi-frequency tomography approach illustrated in FIG. 2 illustrates the general principle that, if each transducer in an array emits a different time-dependent waveform, then a spatial map can be inferred from the time-domain output signal. There are many ways to apply this general principle by choosing a set of time-dependent waveforms for the transducers; as one illustrative example, each transducer in an array could emit an ultrasound wave following a code-division multiple access (CDMA) protocol. However, it could be challenging to generate complicated waveforms for each of hundreds or thousands of ultrasound transducers. For this reason, an especially convenient implementation involves driving each transducer in an array as a pure sinusoid with a different frequency for each transducer. In other words, in the FIG. 2 approach, there is a complicated waveform for each transducer and a very simple (1-to-1) relationship between the scattered light sidebands and the imaging volumes. But that can be reversed so that there is a simple sinusoidal waveform for each ultrasound transducer, but a more complicated and indirect relationship between the sideband amplitudes and phases on the one hand, and the three-dimensional geometry of the target tissue on the other hand.

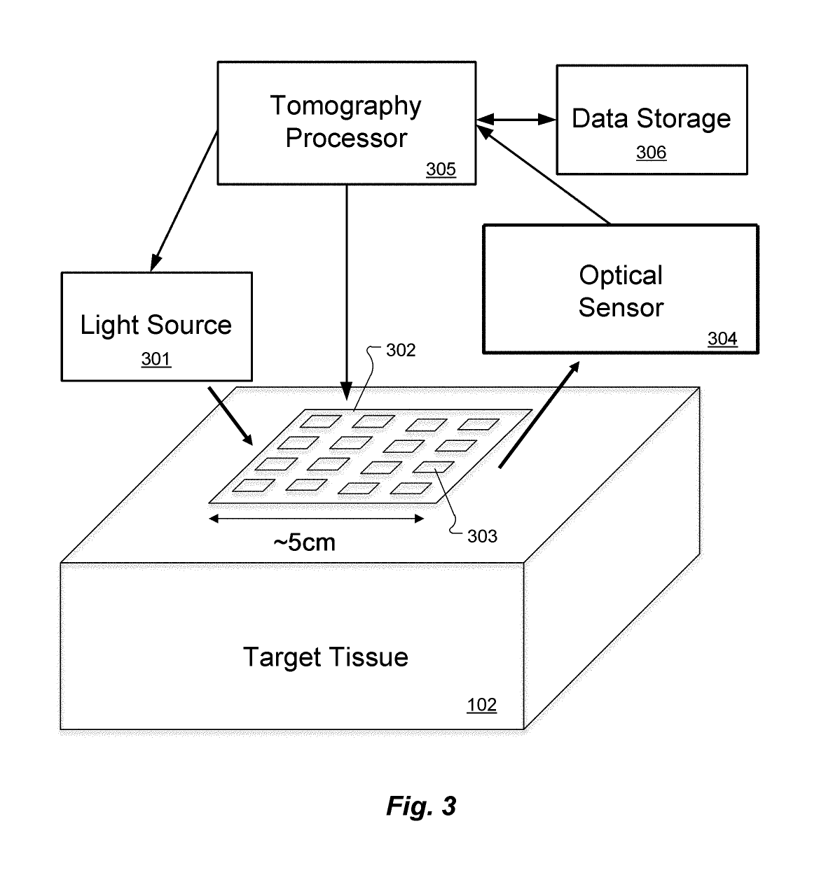

FIG. 3 shows an arrangement for direct multi-frequency ultrasonically-encoded optical tomography of target tissue such as a brain of a patient according to an embodiment of the present invention. Light source 301 (e.g. laser, superluminescent diode, LED, etc.) is configured for generating light input signals to the target tissue 102, for example, to shine light through the skull into the brain. The input light signals from the light source 301 can be sent from a single point, or from several different points, or from a larger-area (defocused) spot. The light source 301 can produce the light input signals non-invasively, if the light is in a wavelength range where the skin and skull are sufficiently transparent or translucent (e.g., red and/or near infrared).

An ultrasound transducer array 302 is configured for placement on the outer surface of the target tissue and has multiple ultrasound transducers 303 each operating at a different ultrasound frequency to generate ultrasound input signals to an imaging volume within the target tissue 102. The ultrasound transducer array 302 might specifically have, for example, 10,000 individual ultrasound transducers 303 on it arranged in a 100×100 square. There may be as few as 10 total ultrasound transducers 303, or as many as 100,000, and they could be arranged in various possible shapes such as a square, circle, annulus, several patches, etc. The spacing between the ultrasound transducers 303 may usefully be related to half the ultrasound wavelength (typically 1 mm or less). A different continuous-wave ultrasound frequency is applied to each individual ultrasound transducer 303. For example, one ultrasound transducer 303 may be vibrating at 5.0000 MHz, another might be at 5.0001 MHz, and so on. For discussion clarity, ultrasound scattering, refraction, etc. will be omitted and it is assumed that each ultrasound transducer 303 creates clean, smooth, outgoing spherical wavefronts in the target tissue 102. (The effects of ultrasound scattering, refraction, etc. are discussed further below.)

An optical sensor 304 is configured for sensing scattered light signals from the imaging volume in the target tissue 102, wherein the scattered light signals include light input signals modulated by acousto-optic interactions with the ultrasound input signals. The optical sensor 304 may specifically include a multi-mode fiber or fiber bundle that takes light scattering out of the target tissue 102 from one or more specific locations and aims it onto a fast detector containing one or more detector elements.

Data storage memory 306 is configured for storing optical tomography software, the scattered light signals, and other system information. An optical tomography processor 305 includes at least one hardware processor coupled to the data storage memory and configured to execute the optical tomography software including instructions to perform spectral analysis of the scattered light signals from the optical sensor 304 to create a three-dimensional image map representing structural and/or functional characteristics of the target tissue 102.

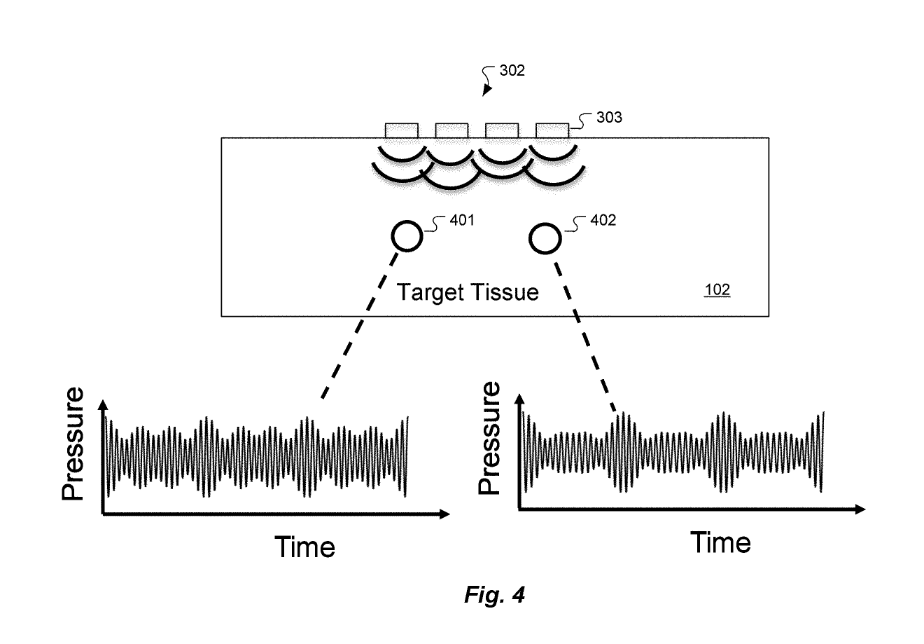

Due to the different ultrasound frequencies, each specific location in the target tissue 102 is subjected to a different time-dependent waveform, distinguished by the relative phase and amplitude of each frequency component. For example, in FIG. 4, the ultrasonic waveforms at two different imaging volumes 401 and 402 are shown (in a schematic, not literal, way). They look different primarily (though not exclusively) because they have different propagation-related phase delays to each of the ultrasound transducers 303. The scattered light in the target tissue 102 is modulated by acousto-optic interactions from the ultrasound signals. For example, a 5.4321 MHz ultrasound transducer causes the light intensity and speckle pattern reaching the optical sensor 304 to oscillate at 5.4321 MHz. Spectral analysis of the scattered light signal should show a peak at 5.4321 MHz, and the amplitude and phase of this peak reflects the amplitude and phase with which the ultrasonic waves from this particular transducer are interacting with the light, in the aggregate.

The spectral analysis performed by the tomography processor 305 includes a post-processing step that converts the amplitude and phase information associated with each ultrasound transducer into the three-dimensional map. This can be thought of (in many ways) as a “holographic reconstruction”. The spectral analysis may be based on a computer model that treats each ultrasound transducer as emitting an ultrasound wave with the phase and amplitude inferred from the amplitude and phase of the corresponding frequency component of the detector data. (The phase may or may not need to be sign-flipped, depending on the sign conventions used.) As all these waves propagate and interfere in the computational simulation, their superposition creates a three-dimensional intensity profile corresponding to the three-dimensional map that is sought. This computer model should include effects such as ultrasound refraction, diffraction, reflection, and scattering (to the extent that these are known). This approach is essentially a matched filter reconstruction, insofar as it is similar to predicting the ultrasound waveform at each point, and evaluating its presence in the output light waveform via a matched filter. Other more sophisticated reconstruction techniques are also possible, including maximum-likelihood or Bayesian-type approaches.

The three-dimensional map produced by the tomography processor 305 reflects the product of local light intensity, local light output probability (i.e. the probability for light at this point to eventually reach the optical sensor 304), and acousto-optic coefficient (which in turn is related to refractive index and other properties of the materials and their configuration).

With reference to the simple example shown in FIG. 4, suppose that acousto-optic interaction occurs in the two indicated small imaging volumes 401 and 402 and nowhere else. Then the detector intensity as a function of time at the optical sensor 304 would appear as a weighted sum of the two waveforms shown. In the holographic reconstruction step of the data analysis, the tomography processor 305 would assign to each ultrasound transducer 303 the amplitude and phase inferred from the corresponding Fourier component of the detected scattered light intensity waveform in a computational acoustic wave propagation simulation. If the ultrasound transducers 303 were hypothetically emitting waves with these amplitudes and phases, they should add coherently to a high intensity at the two small circles of the imaging volumes 401 and 402 and to a much lower intensity everywhere else.

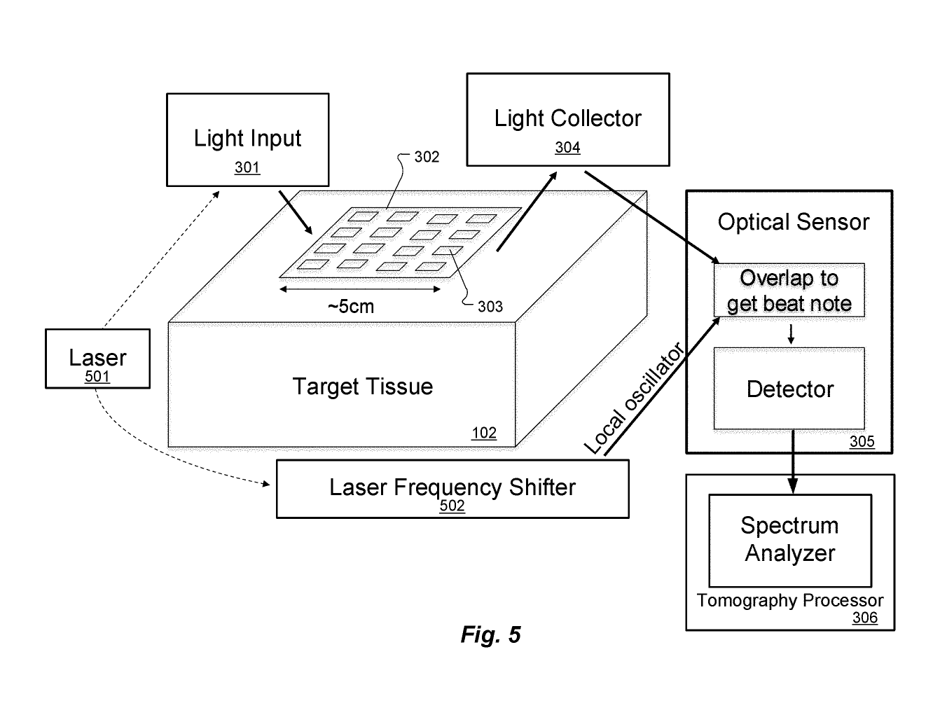

FIG. 5 shows an arrangement for heterodyned optical tomography according to an embodiment of the present invention, which may be a bit more complicated to implement, but may have an improved signal-to-noise ratio (SNR). Laser light from laser 501 is split into two branches (typically fibers). One of these branches is used by the light input 301 to shine light into the target tissue 102 as described above. The other branch of the laser light from laser 501 is frequency shifted by some amount “f shift” by laser frequency shifter 502. This can be done using standard methods such as an acousto-optic modulator, electro-optic modulator, intensity modulator, frequency offset lock, frequency comb techniques, etc. The output light from the laser frequency shifter 502 represents a local oscillator signal. The optical sensor 305 includes a heterodyne light detection arrangement that processes the scatter light from the light collector 304 and the local oscillator signal from the laser frequency shifter 502. This involves overlapping the two light signals onto a fast detector which then sees amplitude modulation related to beat notes. And as above, this is processed by the spectrum analyzer of the tomography processor 306.

Due to acousto-optic interactions, if (for example) 400 THz light goes into the brain, the scattered light exiting is mostly 400 THz, but in the example above it would have sidebands at (400 THz±5.0000 MHz), (400 THz±5.0001 MHz), etc. The spectrum analyzer in the tomography processor 306 should therefore see a strong peak at frequency f shift, with 10,000 pairs of sidebands, one pair for each ultrasound transducer 303. Each pair of sidebands is caused by one particular ultrasound transducer 303, and analysis of the detector output will yield the amplitude and phase with which the ultrasonic waves from this particular ultrasound transducer 303 are interacting with the light, in the aggregate. The post-processing analysis (“holographic reconstruction”) is as above.

In the embodiment in FIG. 5, the local oscillator is a separate light beam, while in the embodiment in FIG. 3, the function of the local oscillator is performed by the non-frequency-shifted light sensed by the optical sensor 305, i.e. the fraction of light that enters and exits the target tissue 102 without interacting with the ultrasound signals. From this consideration, it follows that the heterodyne embodiment in FIG. 5 may be likely to have a higher signal-to-noise ratio than the embodiment in FIG. 3. The explicit local oscillator signal in FIG. 5 can be much stronger because it bypasses the target issue 102 and so is not constrained by safe exposure limits. Moreover, in the embodiment in FIG. 5, various high-sensitivity heterodyne detection techniques can be used (or else used more effectively), such as intensity stabilization of the local oscillator, balanced detection, choosing an f shift that places the sidebands at a frequency most advantageous for high-SNR detection (e.g. low noise and background and systematics), and so forth. On the other hand, the embodiment in FIG. 3 has its own advantages such as simpler hardware and better compatibility with LEDs (as opposed to lasers).

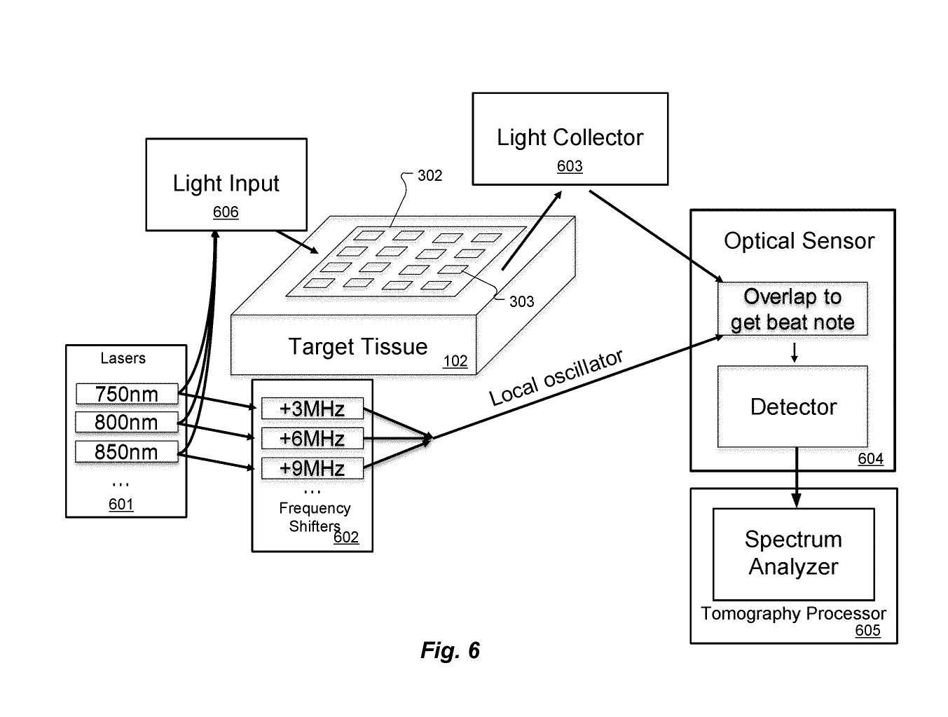

FIG. 6 shows an arrangement for heterodyned multi-frequency optical tomography using multiple wavelength input light simultaneously without sacrificing spatial or temporal resolution and without even needing more than one heterodyne detection module. Lasers 601 create laser light with several different wavelengths for light input 605. The laser light from lasers 601 also is shifted by frequency shifters 602 each by a different frequency in order to create the corresponding local oscillator signal. The light input 606 carries the light signals to the target tissue 102 (either combined or in separate fibers), while the local oscillators are combined and sent to the heterodyne unit within the optical sensor 604. The heterodyne unit sees a complete set of sidebands related to the first wavelength, and, at a different center frequency, a complete set of sidebands related to the second wavelength, and so on. With appropriate frequency choices, these sets of sidebands in the scattered light from the light collector 603 will not overlap, or may only overlap a limited extent, so that they can be separated by the tomography processor 605 in post-processing.

An equivalent functionality could also be accomplished using frequency comb techniques somewhat along the lines of dual-comb spectroscopy. More specifically, the light input would be one frequency comb, and the local oscillators would be a different comb. If the two combs have different teeth spacing, the result would be similar to that in FIG. 6.

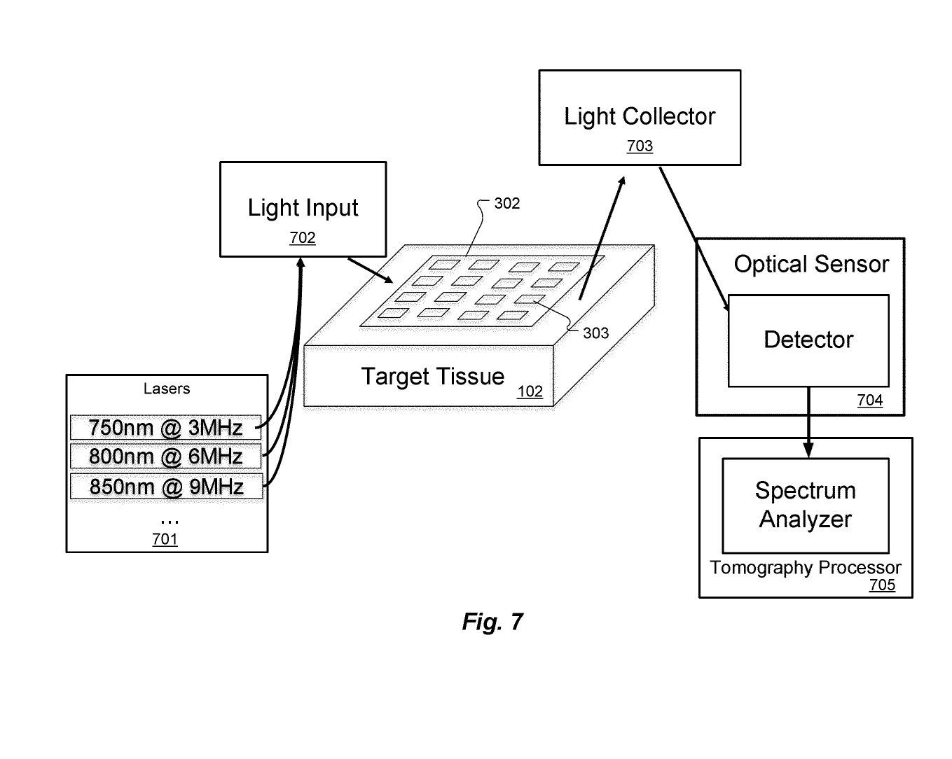

FIG. 7 shows an embodiment for direct multi-frequency optical tomography using multiple wavelength input light without explicit local oscillators or heterodyning. A bank of lasers 701 (or LEDs) is used, and each different wavelength is amplitude-modulated (most simply, switched on and off) at a different rate for delivery to the target tissue 102 by light input 702. This causes sidebands to be duplicated at higher frequencies in the scattered light from the light collector 703 to the optical sensor 704, and hence the tomography processor 705 can extract the different wavelength sidebands with a similar result as in the embodiment in FIG. 6.

One advantageous feature of such arrangements is its speed. New data points are obtained as quickly as the inverse separation between transducer frequencies (e.g. 100 Hz). Partial information is available even faster, though that is more difficult to interpret (but not impossible). And this is a whole three-dimensional image at each 1/(100 Hz) interval, not just one imaging volume (voxel) at a time, and indeed, in multiple-wavelength embodiments, it is a whole three-dimensional image with spatially-resolved spectral information.

This quasi-continuous monitoring can be advantageous for many different applications. One example is mapping brain activation patterns for purposes such as psychological studies, psychiatric diagnoses, brain-machine interfaces for paraplegics, and others. These activation patterns have important high-speed dynamics which usefully can be captured, and for brain-machine interfaces, it is critical to minimize the delay between brain activation and its detection. Another example is that with a high data rate, an embodiment can effectively perform computational correction for motion of the ultrasound transducer array relative to the imaged anatomical features. Implementation would be generally along the lines of the digital image stabilization techniques used in many cameras. Another example is that with a high data rate, a variety of temporal filters can be applied to extract additional information. For example, it is possible to extract just the image or spectral changes that are in synchrony with the pulse rate, by combining measurement data with a heart-rate monitor and then using typical lock-in amplifier-type techniques. Or conversely, the pulse-related changes can be suppressed in the data output. As another example, frequency filtering may enable the sensing of neural activity such as gamma waves.

Another appealing feature is the image resolution, which should be comparable to the ultrasound frequency used, typically 1 mm or less, which is similar to fMRI. Embodiments also provide good signal-to-noise ratio (SNR)—low-noise high-sensitivity heterodyne receivers can be implemented via various known techniques including, for example, balanced detection, local oscillators with high power and intensity stabilization feedback, etc. Embodiments can be implemented at favorably low size, weight, power, and cost. For example, the input light is single-pixel in the sense that a spatial light modulator (SLM) is not required, and the output light is also single-pixel in the sense that there is no detector array strictly required, though it is preferred for improving the sensitivity as discussed below.

It might be useful to include a spatial light modulator (SLM) as part of the light source module, particularly in order to improve the efficiency with which light transmits into (and back out of) the general region being imaged, particularly through the skin and skull. (See “Light finds a way through the maze”, John Pendry, Physics 1, 20 (2008)). The SLM settings could be optimized using existing 3D data available through the device, as this data indirectly indicates the three-dimensional light intensity profile, conveniently including only those photons which eventually reach the optical sensor. While it would increase system complexity, this could provide higher (perhaps dramatically higher) signal-to-noise ratio if input light power is held constant, or reduced light input power for the same signal-to-noise ratio (reducing the risk of skin burning etc.). If a multi-mode fiber is used to carry the input light, the SLM could be located before the light enters the fiber, rather than at the patient's head. An SLM is not the only non-invasive way to increase light transmission through the skin and skull and into a region of interest, which could also involve finely adjusting the optrode angle, and/or position, and/or light wavelength, in order to find a configuration where transmission into the region of interest is higher than usual. Similarly, there could be a spatial light modulator or other adjuster at the output side, in order to increase the efficiency with which light, having exited from the tissue, reaches the small detector.

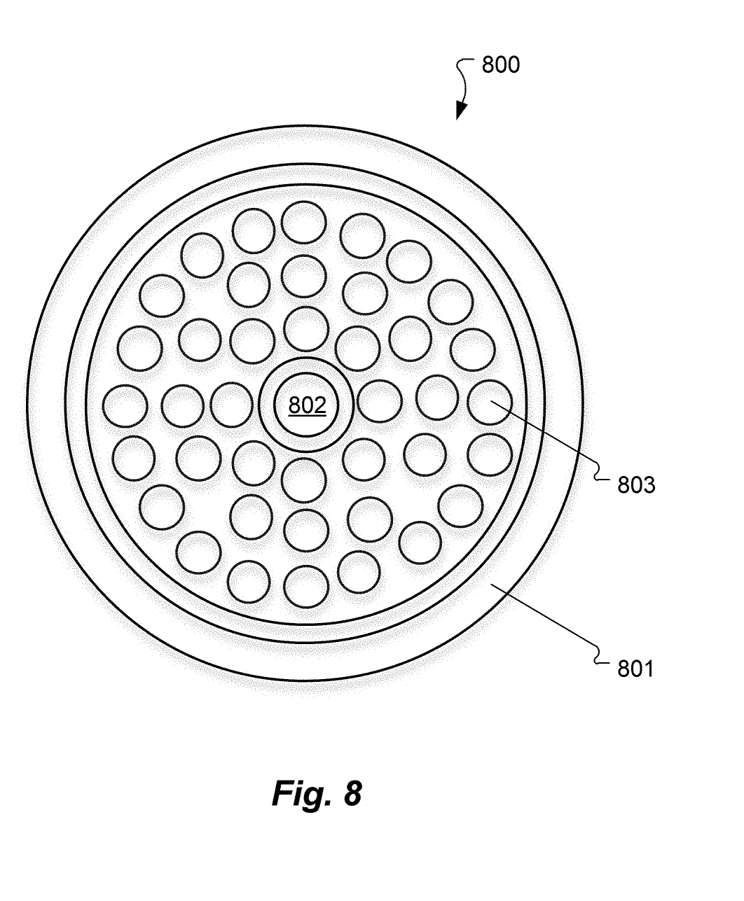

FIG. 8 shows an example of the geometry for an input/sensing device 800 according to an embodiment of the present invention which combines the ultrasound transducer array 803, light input 801, and light collector 802. The light input 801 is formed as a large ring that produces a larger volume of illumination and more uniformity. The light collector 802 extracts the modulated scattered light signals from the center of the input/sensing device 800, and ultrasound transducer array 803 fills the annular space between them and provides the acousto-optic interaction required for position resolution.

When the transducer array is designed, there is some freedom to decide exactly which frequencies go in which transducers, and what phase offset to apply to each transducer. If there were only two transducers with different frequencies, the phase offset would not particularly matter, because their relative phase is changing constantly. But for a larger number of transducers, the phase offsets can have noticeable effects, even if they all have different frequencies. An important consideration when making these decisions is the goal of reducing the ratio of peak instantaneous ultrasound pressure fluctuation to root-mean-square ultrasound pressure fluctuation. This ratio should be minimized everywhere, but especially in the parts of the tissue where the ultrasound power is highest, or where the tissue is most sensitive. If this ratio is reduced, it would allow a higher average ultrasound power without passing safe exposure limits, and hence potentially improve the signal-to-noise ratio. The ratio can be reduced using computational or physical modeling, along with genetic algorithms, machine learning, or other known optimization techniques. Ultrasound-encoded tomography to date has largely (or perhaps entirely) used transducer arrays in which all the transducers have the same time-dependent waveform (apart from a possible phase delay). This limitation makes the device easy to build and operate. But the approach embodied in the present invention uses dozens to thousands of ultrasonic frequencies at once, and so in that sense can be expected to be technically challenging, but there is a high potential reward in improving the sensitivity and performance of any type of ultrasound-encoded tomography.

Overall, the geometrical arrangement of which transducers use which frequency does not matter much under normal imaging conditions; however, this design parameter can have some indirect consequences. For example, pairs of transducers with especially close frequencies—for example 5.4792 MHz vs. 5.4793 MHz—should probably be placed farther apart from each other to reduce undesirable cross-talk via electrical and/or mechanical coupling.

The modulated scattered light output could be tapped at multiple points and/or fed into multiple heterodyne detectors to improve SNR. This might be accomplished as simply as putting multiple fast detectors side-by-side in the same optical sensor unit.

Ultrasound-encoded optical tomography techniques such as discussed herein presents particular challenges in the light detection system. Traditional techniques in ultrasound-encoded optical tomography detection—for example, Fabry-Pérot filters, or two-beam interference in photorefractive crystals, or CMOS detector arrays in conjunction with pulsed light—generally work well only if there is a single time-dependent ultrasound waveform present. But that is not the case for the techniques described herein where ultrasound modulation is present simultaneously over a broad bandwidth, for example, 100 kHz to 10 MHz, and hence the signal is too fast for traditional techniques, even after heterodyning. Alternatively, a single-element fast photodetector may be used, but the resulting signal-to-noise ratio will be sub-optimal because if the detector is large enough to collect substantial optical power, it will receive many different speckles at once, and the modulation of these different speckles will partly cancel each other out.

To avoid such problems, embodiments of the present invention may utilize a many-element fast photodetector array. For example, an array of 10 to 1,000,000 elements, either linear or matrix, is set up such that the size of each detector element is comparable to or larger than the size of one speckle of output light, and such that the overall speed of the detector array is high enough to satisfy the Nyquist criterion for the fastest ultrasound frequency present—for example, faster than 1,000,000-10,000,000 frames per second. Such fast photodetector arrays are available or under development for diverse other applications such as X-ray computed tomography (CT), LIDAR, and fluorescence lifetime imaging systems. The detector elements are frequently either conventional photodetectors (e.g. PIN photodiodes) or Geiger mode avalanche photodiodes. Examples of suitable detector arrays are described in “Fully tileable photodiode matrix for medical imaging by using through-wafer interconnects”, M. Juntunen et al., Nuclear Instruments and Methods in Physics Research A 580 (2007) 1000; and “High frame-rate TCSPC-FLIM using a novel SPAD-based image sensor”, M. Gersbach et al., Proc. SPIE vol. 7780 (2010), 77801H-1, both of which are incorporated herein by reference in their entireties.

Besides increasing the signal-to-noise ratio, a many-element fast photodetector array and appropriate ultrasound source enables an additional operating mode for the system in which photon time-of-flight information is collected concurrently with the ultrasound-encoded position information. Photon time-of-flight information is frequently measured in diffuse optical tomography but not in ultrasound-encoded optical tomography, and carries extra spatial and optical information. For example, this extra spatial and optical information can allow better separation between superficial and deep signals, and can allow more direct measurements of tissue scattering coefficients and other optical properties. In the context of the present invention, the photon time-of-flight information also can help mitigate cross-talk when different parts of the tissue experience similar ultrasound waveforms, and it can also mitigate against the canceling out of ultrasound modulation signals across different optical paths and speckles, mentioned above.

Photon time-of-flight information can be collected either in the time domain or frequency domain. In a time-domain example, a pulsed laser source may pulse at a rate above twice the fastest ultrasound frequency present, for example, it may pulse at 20 MHz for ˜1 MHz ultrasound. Then, for each pulse or each group of pulses, a Geiger-mode avalanche detector array may measure the arrival time of one or more photons striking the pixel, if any. A frequency-domain example could operate similarly, but replacing the 20 MHz pulsed laser with a 20 MHz-repetition-rate swept-source laser, for example.

Typically an optical diode protects the laser light source. And the path lengths of the two optical paths to the heterodyne receiver should be approximately equal. The laser linewidth should be sufficiently narrow and frequency sufficiently stable so as to obtain high-contrast narrow-bandwidth beat notes that are spectrally well separated from each other. For example, a 1 GHz linewidth allows heterodyne beat notes to be visible with up to about 1 foot of optical path length discrepancy between the two paths that are being interfered.

A single instrument could potentially be configured to take measurements using both the modality described above, and also other modalities such as traditional ultrasound, photoacoustic imaging, various fNIRS or diffuse optical tomography techniques, and so on. For example, a traditional ultrasound scan could reveal the acoustic scattering, speed of sound profile, and other parameters that could make the “holographic reconstruction” step (see above) more accurate. As another example, the technique here could be combined with focused ultrasound brain stimulation, in order to not only read but also modify neurological states, including creating complex spatiotemporal excitation and inhibition patterns, with automatic perfect co-registration between the images and excitations. As still another example, the technique here could be combined with high-intensity focused ultrasound in order to destroy a tumor while monitoring progress.

Higher-order acousto-optic interactions could produce extra sidebands or contribute to already existing sidebands in the modulated scatter light, for example, at the ultrasound sum- or difference-frequencies. It may be beneficial to reduce the ultrasound amplitude sufficiently to minimize these types of interactions and so make the data analysis more tractable. However, to the extent that they are present, they could be used in the spectral analysis and could even increase the image resolution (because sum-frequency waves have a shorter wavelength).

A light detection system for massively multi-frequency ultrasound-encoded optical tomography presents particular challenges, particularly due to the requirement of high measurement bandwidth. As an alternative (or complement) to the many-element fast photodetector arrays discussed above, embodiments of the present invention may use photorefractive detection of the ultrasound modulated sum- or difference-frequencies of the scattered light output from the target tissue. This may include a fast high-bandwidth mode for photorefractive detection in which the photorefractive detector light sensor senses scattered light signals at the second-harmonic (or more generally, sum-frequencies) of the ultrasound waveform(s) in the target tissue.

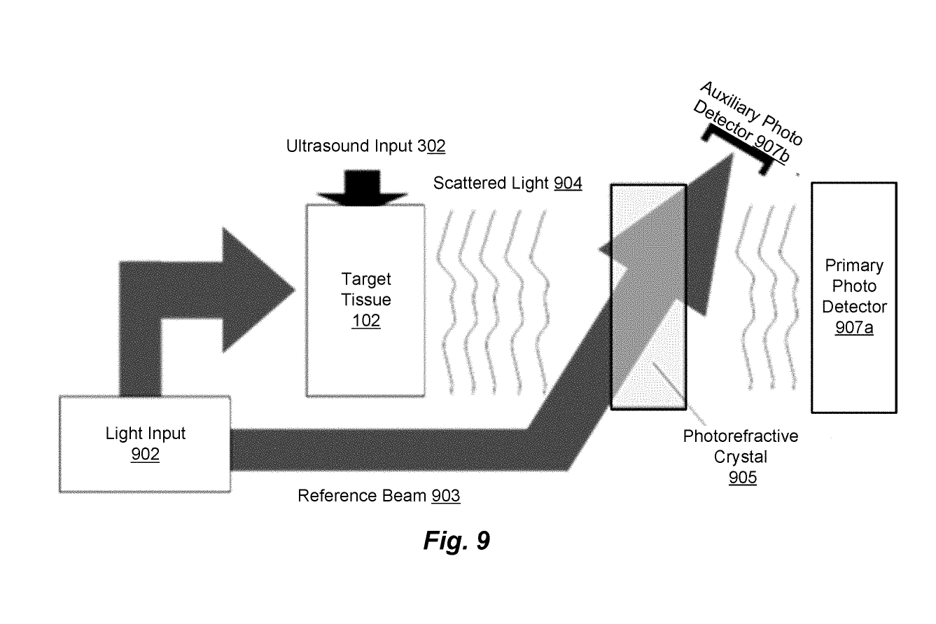

The general idea of photorefractive detection has been described in the literature of the field, for example, in “Theoretical description of the photorefractive detection of the ultrasound modulated photons in scattering media”, M. Gross et al., Optics Express 13, 7097 (2005) (incorporated herein by reference in its entirety). As shown in the schematic block diagram in FIG. 9, the scattered light 904 from the target tissue 102 is overlapped with a reference beam 903 in a photorefractive crystal 905, made, for example, of a gallium arsenide crystal subjected to an applied voltage. Due to photorefractive two-wave mixing, the scattered light measurement by the photorefractive detectors 907a then relates to the amount of ultrasound modulation of the scattered light.

Actual systems are more complicated than the simplified schematic block diagram in FIG. 9, and may involve variations such as large-diameter fibers or fiber bundles that bring scattered light from the target tissue 102 to the photorefractive crystal 905; light scattering out the same side of the target tissue 102 that it enters; laser intensity stabilization; mirrors, isolators, and other optical components, and other components and designs known in the literature. Also, the auxiliary photodetector 907b is more often omitted (i.e. replaced with a beam dump); however the ultrasound modulation is manifested as light intensity modulation with opposite signs for the primary and secondary photodetector, and therefore combining the two signals from the two photodetectors (for example, with a balanced photodetector arrangement) can reduce noise. For massively multi-frequency ultrasound-encoded optical tomography, the reference beam 903 may be at the same optical wavelength as the scattered light 904 (i.e., from the same laser as the light input 902). Other possibilities are known in the literature, including frequency-shifted or phase-modulated reference beams, but these may be less useful for high-bandwidth measurements.

In photorefractive detection, the photodetector registers light modulation both at low frequency—the difference frequencies between ultrasound frequency components present in the target tissue (i.e., the signal related to the envelope of the ultrasound waveform)—and at high frequency—the sum frequencies or second harmonics of the ultrasound frequency components present in the target tissue. Previously, only the former have been recognized and measured in photorefractive systems, however, measuring both the low-frequency and high-frequency components can increase the measurement bandwidth, SNR, and spatial resolution. (To fully measure the high-frequency components, the photodetector and digitizer speed should be at least 4 times the highest ultrasound frequency to allow for Nyquist-rate sampling.)

Since photorefractive detection is inherently nonlinear (i.e., measuring sum and difference frequencies of ultrasound frequencies rather than signals at the ultrasound frequency itself) the corresponding post-processing/reconstruction method performed by the tomography processor is different from the linear “holographic reconstruction” that has been described. One simple and effective starting point is to use matched filters and a grid of N points in the target tissue. At each point, the tomography processing can predict the ultrasound waveform as a pressure-squared-versus-time profile and also as a displacement-squared-versus-time profile, thereby obtaining 2N waveforms. Each of these then can be cross-correlated by the tomography processor with the actual photodetector time-domain signal. The correlation at each point in the measured grid is indicative of the amount of scattered light reaching that point and passing to the photorefractive detector, and also the strength and nature of the acousto-optic interaction at that point in the target tissue. The pressure-squared correlation is specifically caused by the piezo-optic effect, and the displacement-squared correlation is specifically caused by motion of scatterers in the target issue, as discussed, for example, in “Mechanisms of ultrasonic modulation of multiply scattered coherent light: an analytic model”, Lihong V. Wang, Phys. Rev. Lett. 87, 043903 (2001)(incorporated herein by reference in its entirety). These two correlations—with pressure-squared and with displacement-squared—can in general give complementary information. In particular, the ratio of the two is indicative of the ratio between optical mean free path and ultrasound wavelength (see “Mechanisms . . . ” reference above). When the optical mean free path is much shorter than the ultrasound wavelength, the high-frequency parts of the pressure-squared waveform and displacement-squared waveform tend to be equal and opposite (where “high-frequency” means faster than the ultrasound frequencies), and thus a very weak high-frequency response is expected overall. In that case, a high-frequency response can selectively measure areas of unusually little scattering, such as fluid sacs. On the other hand, in the case that the optical mean free path is always much larger than the ultrasound wavelength, the displacement effect is expected to be small, and can often be ignored altogether.

The matched filter approach described above is just one specific non-limiting example, and can be supplemented or replaced by other techniques including deconvolution with the expected point-spread-function, and more generally, incorporating prior knowledge to the tomographic reconstruction (such as continuity of light flow and properties of the tissue), matching data to forward models of light propagation and modulation, accounting for non-localities (i.e. the light modulation depends not only on the pressure at any given point but also the correlations among pressure at nearby points), and so on. Also, the primary photodetector 907a may be split into multiple detector elements for collecting light from different tissue regions, with correspondingly different prior probabilities of taking various possible paths through the tissue.

Photorefractive detection tends to collect information about very low spatial frequencies (from difference frequency or wave-envelope effects) and very high spatial frequencies (from sum frequency or second-harmonic effects). This could leave a gap in between that could cause distracting artifacts in reconstructed images. This gap can be computationally filled in or eliminated by using wide-bandwidth ultrasound (for example, a factor-of-three bandwidth), or exciting multiple ultrasound bands (for example, with one set of transducers designed for around 500 kHz and another set of transducers designed for around 1 MHz bandwidth), or by supplementing photorefractive detection with a different method, such as a fast detector array, which senses ultrasound modulations at the original ultrasound frequency instead of sum or difference frequencies of the ultrasound.

Photorefractive detection is primarily a nonlinear way of detecting a linear acousto-optic modulation, and can thus be distinguished from, for example, the second-harmonic signal in “Nonlinear effects in acousto-optic imaging”, Selb et al., Optics Letters 27, 918 (2002), which is a nonlinear modulation detected in a linear way. In particular, Selb et al. measured both a fundamental and second-harmonic frequency with the same apparatus, whereas a photorefractive detector cannot usually see any appreciable signal at the fundamental ultrasound frequency. To the extent that nonlinear acousto-optic modulation occurs, a photorefractive detector would primarily see it as a fourth harmonic signal. Measuring this fourth-harmonic signal could offer even better spatial resolution for the same ultrasound frequency, or alternatively similar spatial resolution for lower ultrasound frequency. (Lower ultrasound frequency has advantages including deeper penetration and more efficient passage through bones.)

As previously mentioned, the computational ultrasound wave propagation part of the holographic reconstruction process should account for effects such as ultrasound refraction, diffraction, reflection, and scattering, to the extent that these are known. These parameters can be predicted from typical anatomy and/or measured by conventional ultrasound and/or inferred from the three-dimensional image itself. For example, assuming that sound travels at a different speed in the skull than elsewhere, then if the skull thickness profile is estimated incorrectly, it might cause the three-dimensional map to have a warped appearance with straight features appearing wavy. Using such a map, the skull thickness profile could be corrected based on prior knowledge about the shapes of anatomical features. As another example, if a surface has an incorrectly-estimated ultrasound reflection coefficient, then a spurious mirror-reflected copy of features might appear in the three-dimensional map. But this duplication, if recognized, could be used to correct the ultrasound reflection coefficient in the computer model, thus fixing or mitigating the erroneous duplication and so improving the fidelity of the map.

Spectroscopic information can also be obtained by using optical filters to split up different wavelengths, and then having one heterodyne detector for each wavelength. This increases the system complexity but may increase SNR. Spectroscopic information also can be obtained simply by turning one wavelength on, then the next wavelength, etc. But that would impair temporal resolution and perhaps SNR.

There are two prior techniques known in the literature that are somewhat similar to what is described herein in the sense that: (1) three-dimensional spatially-resolved and potentially spectrally-resolved information is obtained, and (2) the resolution is related to ultrasound wavelengths because ultrasound is ultimately used to encode or detect the position. One such approach is known by various terms including ultrasonically-encoded optical tomography, acousto-optic tomography, or ultrasound guide star; see “Time-reversed ultrasonically encoded optical focusing into scattering media”, Xu et al., Nat. Phot. 5, 154 (2011)(incorporated herein by reference in its entirety). Another such approach is known as photoacoustic imaging; see e.g., “Imaging cancer with photoacoustic radar”, Mandelis, Physics Today 70, 42 (2017)(incorporated herein by reference in its entirety). But in their specifics, these two techniques are very different from each other and from the technique described herein.

Photoacoustic imaging uses a very different detailed mechanism, using light to create ultrasonic waves and then detecting that ultrasound with piezo transducers, whereas the embodiments of the present invention described herein use piezo transducers to create ultrasonic waves that modulate light in a way that is detected optically. So in one sense, the two different approaches are opposites. In addition, embodiments of the present invention enable a better signal-to-noise ratio, and allows measuring many wavelengths at once without losing spatial or temporal resolution. Moreover, photoacoustic imaging measures almost purely absorption, whereas embodiments of the present invention are also sensitive to light scattering coefficient and acousto-optic coefficient, which in turn is related to refractive index and other parameters. In this respect, the two different techniques might be complementary, and, as mentioned above, it is conceivable that the same system devices could support both sensing modalities.

Ultrasonically-encoded optical tomography has previously generally used single-frequency ultrasound phased arrays (as in FIG. 1), and therefore image one voxel at a time, and usually also one wavelength at a time. Thus it has been a slow technique. One variant of ultrasonically-encoded optical tomography uses a spatial light modulator (SLM) on the input light. The SLM's phase map is set to focus light of a certain wavelength onto a certain voxel (imaging volume). This phase map is computed using an ultrasound array that focuses sound waves to a particular voxel. In a dynamic living tissue, this variant can be even slower, because it is not only one-voxel and one-wavelength-at-a-time imaging, but also it requires that each of the phase maps be periodically re-measured or re-optimized due to the ever-changing microscopic scattering pattern.

Even though embodiments of the present invention have been discussed in terms of using an SLM on the input light, the purpose and details are quite different. In ultrasound guide star (and other known techniques), the SLM is used to focus light to one voxel, and then get data just about that one voxel, with a separate phase map for each voxel. In embodiments of the present invention, the SLM is provides more light into a relatively large-volume general region (e.g., through the skull into the brain and/or deeper into the brain and/or in the general direction of the light output) much larger than an image voxel. Spatial resolution comes from the ultrasound frequency encoding, not from the SLM, and hence this technique can get images much faster, and with greatly reduced requirements on the speed, size, resolution, and location of the SLM.

Diffuse optical tomography typically just sends light in at one point and collects it at another point. Hence it is far lower resolution than the approach used in embodiments of the present invention, which gets a whole three-dimensional map for each input and output rather than merely one data point. For example, “Mapping distributed brain function and networks with diffuse optical tomography”, Nature Photonics 8, 448 (2014) by Eggebrecht et al. refers to ˜1.5 cm resolution as “high-density diffuse optical tomography”, even though it probes perhaps 3 orders of magnitude larger volume elements than the approach described above for embodiments of the present invention (cm³ instead of mm³). fNIRS (functional near infrared spectroscopy) methods all have similar resolution limitations. Optical coherence tomography (OCT) has higher resolution, but much shallower depth in highly-scattering tissues, since OCT uses photons that only scatter once, whereas the present invention can get good data from photons that have scattered very many times.

Magnetic resonance imaging (MRI) senses different characteristics than light does and also has extremely high size, weight, power, and cost, and is not portable, and generally cannot be used on patients with metal implants (e.g. pacemakers, cochlear implants, etc.). Positron-emission tomography (PET) also observes different characteristics than light does, and has high size, weight, power, and cost, and is not portable, and is sometimes not usable due to the ionizing radiation. Ultrasound (by itself) similarly observes different characteristics than light does. EEG and MEG tend to have far lower resolution than the sub-mm voxels discussed here, and again, they see very different things than light does.

Besides the specific context of ultrasonically-encoded optical tomography as discussed above, the invention can also usefully be embodied in other different specific tomography applications. For example, another category of ultrasound-encoded tomography, which can be called “ultrasound-encoded electrical impedance tomography,” creates high-resolution three-dimensional images of electrical impedance or acousto-electric interaction in a target object, typically at frequencies from DC up to GHz. This category includes acousto-electric tomography (where the probe input signals and the tomography output signals are electric voltages or electric currents on one or more electrodes), acousto-microwave tomography (where the probe input signals and the tomography output signals are each a microwave or radio-frequency electromagnetic field), and magneto-acousto-electric tomography (where the probe input signals and the tomography output signals are a current/voltage on one or more electrodes), and others. These techniques have potential applications in diagnosing injuries, functional brain imaging, functional lung imaging, cancer screening (including breast cancer and liver cancer), image-guided surgery, image-guided radiation therapy, and many other areas. Outside of biology and medicine, it also has potential applications in infrastructure maintenance (e.g. remote corrosion detection), geology (including oil and gas exploration), and other areas.

Yet another category of ultrasound-encoded tomography is called “ultrasound current source density imaging,” which creates high-resolution three-dimensional images of current flow in tissues. It has potential applications in the diagnosis and treatment of epilepsy, heart arrhythmia, and other cardiac, neural, and neuromuscular conditions.

In summary, there is a wide variety of specific ultrasound-encoded tomography techniques which are known and have been demonstrated in the laboratory, but few if any have found practical commercial applications to date. An important reason that these techniques have generally been commercially undeveloped is that the ultrasound is used for essentially only one spatial measurement at a time. Most commonly, one small volume (“voxel”) in three-dimensional space is imaged at a time. However, there are variants (such as “Ultrafast acousto-optic imaging with ultrasonic plane waves”, Laudereau et al., Optics Express 24, 3774 (2016)) in which the spatial interrogation region takes a different shape besides a point. But regardless of these details, there is only one spatial measurement at a time, and therefore there is naturally a tradeoff wherein either the scan is very slow (and hence inconvenient, vulnerable to motion blur, and incapable of seeing dynamic processes), or the signal-to-noise ratio is very low (from inadequate integration time), or the integration volume is purposefully shrunk, or the spatial resolution is purposefully degraded from its inherent hardware limit (as in Laudereau et al. above).

Embodiments of the present invention such as those discussed above can significantly improve the speed, and/or sensitivity of ultrasound-encoded tomography, and can be useful in any or all of the numerous applications listed above as well as others omitted for brevity.

Embodiments of the invention may be implemented in part in any conventional computer programming language such as VHDL, SystemC, Verilog, ASM, etc. Alternative embodiments of the invention may be implemented as pre-programmed hardware elements, other related components, or as a combination of hardware and software components.

Embodiments can be implemented in part as a computer program product for use with a computer system. Such implementation may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or analog communications lines) or a medium implemented with wireless techniques (e.g., microwave, infrared or other transmission techniques). The series of computer instructions embodies all or part of the functionality previously described herein with respect to the system. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). Of course, some embodiments of the invention may be implemented as a combination of both software (e.g., a computer program product) and hardware. Still other embodiments of the invention are implemented as entirely hardware, or entirely software (e.g., a computer program product).

Although various exemplary embodiments of the invention have been disclosed, it should be apparent to those skilled in the art that various changes and modifications can be made which will achieve some of the advantages of the invention without departing from the true scope of the invention.

What is claimed is:

1. A computer-implemented system for multi-frequency ultrasonically-encoded optical tomography of a target object having an outer surface, the system comprising:
one or more probe inputs configured for generating optical probe input signals to the target object;
an ultrasound transducer array configured for placement on the outer surface of the target object and having a plurality of ultrasound transducers each generating a different time-dependent waveform to form a plurality of ultrasound input signals to a target probe volume within the target object;
a photorefractive crystal configured for mixing scattered light output signals from the target probe volume with an optical reference beam input to the photorefractive crystal to produce optical tomography output signals including ultrasound sum frequencies components;
a photodetector configured for sensing the optical tomography output signals from the photorefractive crystal;
data storage memory configured for storing optical tomography software, the optical tomography output signals, and other system information;
a tomography processor including at least one hardware processor coupled to the data storage memory and configured to execute the optical tomography software including instructions to perform acousto-optic tomography analysis of the optical tomography output signals including the ultrasound sum frequencies components to create a three-dimensional object map representing structural and/or functional characteristics of the target object.

2. The system according to claim 1, further comprising:
an auxiliary photodetector configured for sensing a reference beam output signal from the photorefractive crystal characterized by light modulation opposite in sign from the optical tomography output signals and including ultrasound sum frequencies components; and
wherein the optical tomography software executed by the tomography processor includes instructions to perform acousto-optic tomography analysis of the ultrasound sum frequencies components of the optical tomography output signals and the reference beam output signal.

3. The system according to claim 1, further comprising:
an optical fiber arrangement configured for communicating the scattered light output signals from the target probe volume to the photorefractive crystal.

4. The system according to claim 1, wherein the different time-dependent waveforms represent different ultrasound frequencies.

5. The system according to claim 1, wherein the optical reference beam input is from the one or more probe inputs generating the optical probe input signals.

6. The system according to claim 1, wherein the optical tomography output signals further include ultrasound difference frequencies components, and wherein the optical tomography software executed by the tomography processor includes instructions to perform acousto-optic tomography analysis of the ultrasound sum frequencies components and the ultrasound difference frequencies components of the optical tomography output signals.

7. The system according to claim 1, wherein the photorefractive detector elements are configured for operation at a speed at least four times greater than the greatest ultrasound frequency.

8. The system according to claim 1, wherein the optical tomography software executed by the tomography processor includes instructions to perform acousto-optic tomography analysis using matched filters to create the three-dimensional object map.

9. The system according to claim 1, wherein the optical tomography software executed by the tomography processor includes instructions to perform acousto-optic tomography analysis using ultrasound waveform predictions that include a pressure-squared-versus-time profile and a displacement-squared-versus-time profile for each sampling point.

10. The system according to claim 1, wherein the optical tomography software executed by the tomography processor includes instructions to perform acousto-optic tomography analysis using supplemental optical tomography output signals having ultrasound components at the ultrasound frequencies of the ultrasound input signals.

11. The system according to claim 1, wherein the photorefractive crystal is made of gallium arsenide.

12. The system according to claim 1, wherein the ultrasound sum frequencies components include second-harmonic frequency components.

13. A computer-implemented method employing at least one hardware implemented computer processor for multi-frequency ultrasonically-encoded optical tomography of a target object having an outer surface, the method comprising:
operating the at least one hardware processor to execute program instructions for:
generating optical probe input signals to the target object;
operating an ultrasound transducer array placed on the outer surface of the target object and having a plurality of ultrasound transducers each generating a different time-dependent waveform to form a plurality of ultrasound input signals to a target probe volume within the target object;
mixing scattered light output signals from the target probe volume with an optical reference beam input to a photorefractive crystal so as to produce optical tomography output signals including ultrasound sum frequency components;
sensing the optical tomography output signals from the photorefractive crystal with a photodetector;
performing acousto-optic tomography analysis of the optical tomography output signals including the ultrasound sum frequency components to create a three-dimensional object map representing structural and/or functional characteristics of the target object.

14. The method according to claim 13, further comprising:
sensing a reference beam output signal from the photorefractive crystal characterized by light modulation signals opposite in sign from the optical tomography output signals and including ultrasound sum frequencies components; and
wherein the acousto-optic tomography analysis is of the ultrasound sum frequencies components of the optical tomography output signals and the reference beam output signal.

15. The method according to claim 13, further comprising:
communicating the scattered light output signals from the target probe volume to the photorefractive crystal with an optical fiber arrangement.

16. The method according to claim 13, wherein the different time-dependent waveforms represent different ultrasound frequencies.

17. The method according to claim 12, wherein the optical reference beam input is generated by one or more probe inputs generating the optical probe input signals.

18. The method according to claim 13, wherein the optical tomography output signals further include ultrasound difference frequencies components, and wherein the acousto-optic tomography analysis is of the ultrasound difference frequencies components and the ultrasound sum frequencies components of the optical tomography output signals.

19. The method according to claim 13, wherein the photorefractive detector elements are configured for operation at a speed at least four times greater than the greatest ultrasound frequency.

20. The method according to claim 13, wherein the acousto-optic tomography analysis uses matched filters to create the three-dimensional object map.

21. The method according to claim 13, wherein the acousto-optic tomography analysis uses ultrasound waveform predictions that include a pressure-squared-versus-time profile and a displacement-squared-versus-time profile for each sampling point.

22. The method according to claim 13, wherein the acousto-optic tomography analysis uses supplemental optical tomography output signals having ultrasound components at the ultrasound frequencies of the ultrasound input signals.

23. The method according to claim 13, wherein the photorefractive crystal is made of gallium arsenide.

24. The method according to claim 13, wherein the ultrasound sum frequencies components include second-harmonic frequency components.
